(19) United States
(12) Patent Application Publication
(10) Pub. No.: US 2024/0100141 A1
(43) Pub. Date: Mar. 28, 2024

(54) T CELL-DIRECTED ANTI-CANCER VACCINES AGAINST COMMENSAL VIRUSES FOR TREATING MUCOSAL CARCINOMAS

(71) Applicant: The General Hospital Corporation, Boston, MA (US)

(72) Inventor: Shadmehr Demehri, Charlestown, MA (US)

(21) Appl. No.: 18/273,466

(22) PCT Filed: Jan. 21, 2022
(86) PCT No.: PCT/US2022/013228

§ 371 Date: Jul. 20, 2023

Related U.S. Application Data
(60) Provisional application No. 63/140,159, filed on Jan. 21, 2021.

(51) Int. Cl. A61K 39/12; A61P 35/00; C07K 14/005; C12N 7/00

(52) U.S. Cl. CPC A61K 39/12; A61P 35/00; C07K 14/005; C12N 7/00; C12N 2710/20022; C12N 2710/20034; C12N 2710/20061; C12N 2710/20071

(57) ABSTRACT

Immune-based approaches to treat and reduce the risk of cancer of mucosal tissues by boosting T cell immunity against commensal HPVs.

### CLAIM OF PRIORITY

This application claims the benefit of U.S. Provisional Patent Application Ser. No. 63/140,159, filed on Jan. 21, 2021. The entire contents of the foregoing are hereby incorporated by reference.

### TECHNICAL FIELD

Immune-based approaches to treat and prevent mucosal cancers by boosting T cell immunity against commensal HPVs.

### BACKGROUND

Mucosal carcinomas including mucosal squamous cell carcinoma (mSCC) constitute a common type of cancer affecting the upper aerodigestive tract, anus and cervix¹. High-risk human papillomavirus (HPV) is the cause of a subset of head and neck cancer (HNC), anal and cervical cancers.

### SUMMARY

As shown herein, vaccines based on commensal HPVs are beneficial in protecting against mucosal cancers.

Thus, provided herein are methods of treating, or reducing the risk of developing, mucosal cancers in a subject, the method comprising administering to the subject an effective amount of a composition comprising (i) a plurality of antigenic peptides each comprising a sequence of 9-30 amino acids derived from proteins from commensal human papilloma viruses or (ii) a plurality of live or live attenuated commensal human papilloma viruses, and a T cell adjuvant that increases T cell response to the antigenic peptides. Additionally provided are the compositions described herein for use in a method of treating, or reducing the risk of developing, mucosal cancer in a subject.

In some embodiments, the subject has an increased risk of developing mucosal cancer or is immunocompromised, e.g., as a result of an acquired immunodeficiency, primary immunodeficiency, or an organ transplant.

In some embodiments, the commensal human papilloma viruses are low risk α-HPV, β-HPV, γ-HPV, and/or μ-HPV strains, e.g., the commensal human papilloma viruses are β-HPV and/or γ-HPV strains listed in Table A.

In some embodiments, the plurality of antigenic peptides comprises peptides derived from one or more E1, E2, E4, E5, E6 or E7 proteins.

In some embodiments, the plurality of antigenic peptides comprises peptides derived from proteins from a plurality of commensal human papilloma viruses.

In some embodiments, the compositions comprise at least 200 peptides each having a unique sequences, e.g., comprising a plurality of peptides for each unique sequence.

In some embodiments, the T cell adjuvant comprises one or more of nanoparticles that enhance T cell response; poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminium salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant). In some embodiments, the T cell adjuvant comprises topical imiquimod and/or topical 5-fluorouracil and/or topical calcipotriene (calcipotriol), e.g., in combination with 5-fluorouracil.

In some embodiments, the mucosal cancer is a cancer of the oral and sinonasal mucosa, optionally mucosal squamous cell carcinoma (mSCC), e.g., of the tongue; head and neck cancer (HNC); a cancer of the mucosa of the upper respiratory tract; or a cancer of the genitourinary tract, optionally anal cancer, cervical cancer, or a cancer of the vulva, vestibule, vagina, perineum, or perianal tissue.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Papillomavirus colonization protects against carcinogen-induced tongue cancer. (FIG. 1A) Time to tumor onset on MmuPV1- versus sham-infected tongue of immunocompetent p53^{+/-} mice after treatment with DMBA/4NQO carcinogenesis protocol (log-rank test). (FIG. 1B) Survival of MmuPV1- versus sham-infected immunocompetent p53^{+/-} mice after treatment with DMBA/4NQO carcinogenesis protocol (log-rank test). Note that the burden of oral lesions induced by carcinogens is the cause of weight loss and mortality in the animals. (FIG. 1C) Representative H&E-stained images of MmuPV1- (top) and sham-infected (bottom) tongue treated with DMBA/4NQO. Red arrow points to the invasive mSCC in sham-infected tongue. n=15 in MmuPV1 and n=16 in sham group.

FIGS. 2A-B. Papillomavirus colonization protects against the expansion of carcinogen-induced mutant Trp53 clones in tongue epithelium. (FIG. 1A) The size of mutant Trp53 clones in MmuPV1- versus sham-infected tongue of immunocompetent p53^{+/-} mice after treatment with DMBA/4NQO carcinogenesis protocol (Mann Whitney U test). (FIG. 1B) Representative p53-stained images of MmuPV1- (top) and sham-infected (bottom) tongue treated with DMBA/4NQO. Red arrow points to the mutant Trp53 clones in MmuPV1- and sham-infected tongue.

### DETAILED DESCRIPTION

High-risk HPV vaccines that are directed at humeral immunity reduce the risk of mSCC by blocking α-HPV infection. However, an effective modality to prevent the large majority of HNCs caused by smoking and mSCCs in individuals who are already infected with high-risk HPVs is lacking. Treatments for mSCC include surgery and radiation for localized disease, and chemotherapy, targeted therapy and immunotherapy for unresectable and metastatic cancers. In addition to their side effects, the treatments for mSCC represents a rising public health challenge. Therefore, new technologies to prevent and treat mSCC are urgently needed.

High-risk α-HPVs are known to induce oropharyngeal mSCCs²; however, carcinogen-induced HNCs constitute more than 70% of mSCCs that affect the upper aerodigestive tract³. Importantly, low-risk commensal HPVs are ubiquitously found in the upper aerodigestive tract, anal canal and reproductive tract^{4,5}. Although they do not make any oncogenic contribution to mSCC development, the impact of immunity against commensal HPVs on mSCC development is unexplored.

Building on the potential of commensal virome for cancer prevention and therapy, described herein is a live commensal HPV vaccine strategy to boost antiviral T cell immunity in the cutaneous and mucosal sites colonized by commensal HPVs to reduce the risk of cancer development and treat early SCCs that contain active virus. As shown herein, the cancer-protective role of commensal HPVs and the immunity against them in the skin by studying patients' skin and cancer samples as well as a novel mouse papillomavirus (MmuPV1) model, which has similar properties to low-risk commensal HPVs. In stark contrast to prevailing dogma, it was discovered that MmuPV1 skin colonization of immunocompetent mice had no oncogenic effect but instead protected the skin from chemical as well as UV skin carcinogenesis in a CD8⁺ T cell-dependent manner. Furthermore, the adoptive transfer of memory T cells from skin draining lymph nodes of the immune mice (i.e., no wart) rendered immunity to wart-bearing mice and provided them with protection against skin carcinogenesis⁶. As described herein, commensal HPV vaccines can also be used for cancer prevention and therapy in mucosal sites, as papillomavirus colonization of the oral cavity protected immunocompetent hosts from mSCC.

T Cell-Based Vaccines Against β-HPVs

Described herein are compositions that can be used to induce a T cell-based immune response against β-HPVs, thereby reducing the risk that the subject will develop mucosal cancer. The vaccines induce T cell immunity against commensal viruses that have already infected the tissue, with the goal not to prevent or eliminate the infection but rather to use of the virus presence in all cells to boost the detection of early cancerous clones and their elimination by T cells. Current high-risk HPV vaccines for cervical and head and neck cancer prevention are meant to prevent infection in the first place and have minimal efficacy in individuals already infected with the virus.

### Antigenic Peptides

In some embodiments, the present compositions include a plurality of antigenic peptides derived from (i.e., comprising a fragment of, i.e., consecutive amino acids from) proteins, e.g., E1, E2, E6, or E7 proteins, from commensal human papilloma viruses, e.g., low risk cutaneotropic α-HPV, β-TPV, γ-HPV and/or μ-HPV strains such as those listed in Table A. In some embodiments, the compositions do not include peptides derived from HPV types that are associated with cancer, e.g., high-risk HPVs such as HPV16 or 18. See, e.g., Ma et al., J Virol. 2014 May; 88(9): 4786-4797; Doorbar et al., Rev Med Virol. 2015 March; 25(Suppl Suppl 1): 2-23; Doorbar et al., The biology and life-cycle of human papillomaviruses. Vaccine 2012; 30(Suppl 5): F55-F70; de Villiers, Virology 2013; 445(1-2): 2-10; and U.S. Pat. No. 8,652,482, which are incorporated herein by reference. Other commensal HPV types that are not “high-risk”-HPVs can be used; Table A is an exemplary but not exhaustive list; other HPVs in normal skin sand mucosal sites are also known, see, e.g., Ma, Journal of virology 2014: 88; 4786-4797.

TABLE A

| HPV | HPV | HPV | | | | | | |
|---|---|---|---|---|---|---|---|---|
| Genus | Species | Type | E1 | E2 | E4 | E5 | E6 | E7 |
| α | 4 | HPV2 | ABO14922.1 | ABO14923.1 | ABO14924.1 | — | ABO14920.1 | ABO14921.1 |
| α | 2 | HPV3 | CAA52471.1 | CAA52472.1 | — | — | CAA52469.1 | — |
| α | 8 | HPV7 | CAA52478.1 | CAA52479.1 | — | — | NP_041854.1 | CAA52477.1 |
| α | 2 | HPV10 | CAA52491.1 | CAA52492.1 | — | — | CAA52489.1 | CAA52490.1 |
| α | 4 | HPV27 | CAA52538.1 | CAA52539.1 | BAE16267.1 | — | CAA52536.1 | CAA52537.1 |
| α | 2 | HPV28 | AAA79424.1 | AAA79425.1 | P51896.2 | — | AAA79422.1 | AAA79423.1 |
| α | 4 | HPV57 | CAA39432.1 | CAA39433.1 | CAA39434.1 | — | CAA39430.1 | CAA39431.1 |
| α | 2 | HPV77 | CAA75465.1 | CAA75466.1 | CAA75469.1 | — | CAA75463.1 | CAA75464.1 |
| β | 1 | HPV5 | AFL02855.1 | AFL02856.1 | AAA46987.1 | AAA46988.1 | CAA52693.1 | CAA52694.1 |
| β | 1 | HPV8 | P06420.1 | P06422.1 | P06425.2 | — | P06428.1 | P06430.1 |
| β | 2 | HPV9 | CAA52485.1 | CAA52486.1 | — | — | CAA52483.1 | CAA52484.1 |
| β | 1 | HPV12 | CAA52498.1 | CAA52499.1 | — | — | CAA52496.1 | CAA52497.1 |
| β | 1 | HPV14 | CAA52502.1 | CAA52503.1 | S65692 | — | P28830.1 | BAA09114.1 |
| β | 2 | HPV15 | CAA52508.1 | CAA52509.1 | — | — | CAA52506.1 | CAA52507.1 |
| β | 2 | HPV17 | CAA52514.1 | CAA52515.1 | AFL02863.1 | — | CAA52512.1 | CAA52513.1 |
| β | 1 | HPV19 | CAA52520.1 | CAA52521.1 | — | — | P36806.1 | CAA52519.1 |
| β | 1 | HPV20 | ARV85572.1 | ARV85573.1 | ARV85574.1 | — | ARV85578.1 | ARV85575.1 |
| β | 1 | HPV21 | AAA79396.1 | AAA79397.1 | AAA79398.1 | — | AAA79394.1 | AAA79395.1 |
| β | 2 | HPV22 | AAA79403.1 | AAA79404.1 | AAA79405.1 | — | AAA79401.1 | AAA79402.1 |
| β | 2 | HPV23 | AAA79410.1 | AAA79411.1 | AAA79412.1 | — | AAA79408.1 | AAA79409.1 |
| β | 1 | HPV24 | AAA79417.1 | AAA79418.1 | AAA79419.1 | — | AAA79415.1 | AAA79416.1 |
| β | 1 | HPV25 | CAA52526.1 | CAA52527.1 | — | — | CAA52524.1 | CAA52525.1 |
| β | 1 | HPV36 | AAA79438.1 | AAA79439.1 | AAA79440.1 | — | AAA79436.1 | AAA79437.1 |
| β | 2 | HPV37 | AAA79445.1 | AAA79446.1 | AAA79447.1 | — | AAA79443.1 | AAA79444.1 |
| β | 2 | HPV38 | AAA79452.1 | AAA79453.1 | AAA79454.1 | — | AAA79450.1 | AAA79451.1 |
| β | 1 | HPV47 | AAA46978.1 | AAA46979.1 | AAA46980.1 | — | AAA46976.1 | AAA46977.1 |
| β | 3 | HPV49 | CAA52581.1 | CAA52582.1 | — | — | CAA52579.1 | CAA52580.1 |
| β | 3 | HPV75 | CAA75451.1 | CAA75452.1 | CAA75455.1 | — | CAA75449.1 | CAA75450.1 |
| β | 3 | HPV76 | CAA75458.1 | CAA75459.1 | CAA75462.1 | — | CAA75456.1 | CAA75457.1 |
| β | 2 | HPV80 | CAA75472.1 | CAA75473.1 | CAA75474.1 | — | CAA75470.1 | CAA75471.1 |
| β | 4 | HPV92 | NP_775307.1 | NP_775308.1 | NP_775309.1 | — | NP_775305.1 | NP_775306.1 |
| β | 1 | HPV93 | AAQ88287.1 | AAQ88283.1 | AAQ88288.1 | — | AAQ88286.1 | AAQ88282.1 |
| β | 5 | HPV96 | NP_932321.1 | NP_932322.1 | NP_932323.1 | — | NP_932319.1 | NP_932320.1 |
| β | 2 | HPV107 | ABN79869.1 | ABN79870.1 | ABN79871.1 | — | ABN79867.1 | ABN79868.1 |
| β | 2 | HPV110 | ACC78265.1 | ACC78267.1 | ACC78266.1 | — | ACC78263.1 | ACC78264.1 |
| β | 2 | HPV111 | ACC78272.1 | ACC78274.1 | ACC78273.1 | — | ACC78270.1 | ACC78271.1 |
| β | 1 | HPV129 | ADR77929.1 | ADR77930.1 | ADR77931.1 | — | ADR77932.1 | ADR77933.1 |
| β | 1 | HPV151 | CBK38955.1 | CBK38956.1 | CBK38957.1 | — | CBK38953.1 | CBK38954.1 |
| β | 2 | HPV164 | AFV27105.1 | AFV27106.1 | AFV27107.1 | — | AFV27103.1 | AFV27104.1 |
| γ | 1 | HPV4 | CAA50159.1 | CAA50160.1 | CAA50161.1 | — | CAA50157.1 | CAA50158.1 |
| γ | 22 | HPV30 | ALT54645.1 | ALJ32745.1 | ALJ32746.1 | — | ALT54643.1 | ALT54644.1 |
| γ | — | HPV33 | ACL12328.1 | ACL12329.1 | ACL12330.1 | ACL12331.1 | AGM34423.1 | AGM34459.1 |
| γ | 22 | HPV35 | ARQ82604.1 | ARQ82605.1 | ARQ82607.1 | ARQ82606.1 | ASA39832.1 | ASA39833.1 |
| γ | 3 | HPV48 | NP_043418.1 | NP_043419.1 | NP_043420.1 | — | NP_043416.1 | NP_043417.1 |
| γ | 2 | HPV50 | NP_043425.1 | NP_043426.1 | NP_043427.1 | — | NP_043423.1 | NP_043424.1 |
| γ | 4 | HPV60 | NP_043439.1 | NP_043440.1 | NP_043441.1 | — | NP_043437.1 | NP_043438.1 |
| γ | 1 | HPV65 | CAA50173.1 | CAA50174.1 | CAA50175.1 | — | CAA50171.1 | CAA50172.1 |
| γ | 5 | HPV88 | ABR20504.1 | ABR20505.1 | ABR20506.1 | — | ABR20502.1 | ABR20503.1 |
| γ | 1 | HPV95 | CAF05704.1 | CAF05705.1 | CAF05706.1 | — | CAF05702.1 | CAF05703.1 |
| γ | — | HPV101 | AAZ39508.1 | AAZ39509.1 | AAZ39510.1 | — | — | AAZ39507.1 |
| γ | 14 | HPV103 | AAZ39486.1 | AAZ39487.1 | AAZ39488.1 | — | — | AAZ39485.1 |
| γ | 9 | HPV121 | ADH29807.1 | ADH29808.1 | ADH29809.1 | — | ADH29805.1 | ADH29806.1 |
| γ | 15 | HPV135 | AEM24595.1 | AEM24596.1 | AEM24597.1 | — | AEM24593.1 | AEM24594.1 |
| γ | — | HPV161 | AFV27124.1 | AFV27125.1 | — | — | AFV27122.1 | AFV27123.1 |
| γ | 8 | HPV164 | AFV27105.1 | AFV27106.1 | AFV27107.1 | — | AFV27103.1 | AFV27104.1 |
| γ | 16 | HPV186 | ANG08953.1 | ANG08954.1 | — | — | ANG08951.1 | ANG08952.1 |
| γ | 2 | HPV200 | AKP16336.1 | AKP16337.1 | — | — | AKP16334.1 | AKP16335.1 |
| μ | 1 | HPV1 | P03111-1 | P03118-1 | P06923-1 | — | NP_040305.1 | NP_040307.1 |
| μ | — | HPV63 | CAA50166.1 | CAA50167.1 | CAA50168.1 | — | CAA50164.1 | CAA50165.1 |

The peptides can be derived from any antigenic protein in the virus; in some embodiments, the peptides are derived from an E1, E2, E4, E5, E6 or E7 protein. Sequences for these proteins in a number of commensal strains are provided. In some embodiments, at least 50 or more, 100, 150, 200, 250, 300, 350, 400, 450, 500, or more different peptides (i.e., peptides having different sequences) are included in the compositions. In some embodiments, at least 50 or more, 100, 150, 200, 250, 300, 350, 400, 450, 500, or more different peptides from each virus strain are included in the compositions, and peptide from two or more virus strains are included.

In some embodiments, the peptides are of a length that is optimized for MHCI/MHCII presentation, e.g., 9-30 amino acids, e.g., 12-25, 12-18, 12-16, 13-16, 14-16, or 15 amino acids. The sequences of the peptides can be synthetic long overlapping peptides, e.g., identified, e.g., bioinformatically to predict antigenicity and/or generated using a moving window of overlapping peptides to cover the entire protein, e.g., 15 amino acid peptides with 10 amino acid overlap (similar to the “gene walk” methods used to identify optimal antisense oligonucleotides). In some embodiments, overlapping synthetic long peptides (SLPs) are used (Zom et al., Cancer Immunol Res. 2014 August; 2(8):756-64). The compositions can include a plurality of peptides derived from one or more (e.g., a plurality of) different virus strains. The peptides are preferably synthetic peptides; methods for synthesizing peptides are known in the art, including solution-phase techniques and solid-phase peptide synthesis (SPPS). See, e.g., Petrou and Sarigiannis, Ch. 1—Peptide synthesis: Methods, trends, and challenges, In: Editor(s): Sotirios Koutsopoulos, Peptide Applications in Biomedicine, Biotechnology and Bioengineering, Woodhead Publishing, 2018, pages 1-21; and Chandrudu et al., Molecules 2013, 18, 4373-4388.

### Antigenic Proteins

In some embodiments, the present compositions can include a plurality of proteins, e.g., virus-like particles containing of E1, E2, E6, or E7 proteins from commensal human papilloma viruses, e.g., low risk α-HPV, β-HPV, γ-HPV and/or μ-HPV strains such as those listed in Table A (see, e.g., Yang et al., Virus Res 231, 148-165 (2017); Hancock et al., Therapeutic HPV vaccines. Best Pract Res Clin Obstet Gynaecol 47, 59-72 (February 2018); Joh et al., Exp Mol Pathol.; 93(3):416-21 (2012)).

### Nucleic Acid-Based Vaccines

In some embodiments, the present compositions can include a plurality of DNA plasmids and/or RNA replicons that contain nucleotide sequences to express proteins or antigenic peptides derived from (i.e., comprising a fragment of, i.e., consecutive amino acids from) proteins, e.g., E1, E2, E6, or E7 proteins, from commensal human papilloma viruses, e.g., low risk α-HPV, β-HPV, γ-HPV and/or μ-HPV strains such as those listed in Table A (see, e.g., Yang et al., Virus Res 231, 148-165 (2017); Hancock et al., Therapeutic HPV vaccines. Best Pract Res Clin Obstet Gynaecol 47, 59-72 (2018)).

### Live Vector-Based Vaccines

In some embodiments, the present compositions can include a plurality of viral vectors that are engineered to express proteins or antigenic peptides derived from (i.e., comprising a fragment of, i.e., consecutive amino acids from) proteins, e.g., E1, E2, E6, or E7 proteins, from commensal human papilloma viruses, e.g., low risk α-HPV, β-HPV, γ-HPV and/or μ-HPV strains such as those listed in Table A (see, e.g., Yang et al., Virus Res 231, 148-165 (2017); Hancock et al., Therapeutic HPV vaccines. Best Pract Res Clin Obstet Gynaecol 47, 59-72 (2018)).

Viral vectors for use in the present methods and compositions include recombinant retroviruses, adenovirus, adeno-associated virus, alphavirus, and lentivirus.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in numerous tissues including the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficient and selectivity, e.g., biotinylated AAV vectors, directed molecular evolution, self-complementary AAV genomes and so on. In some embodiments, AAV1 is used.

Alternatively, retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Alphaviruses can also be used. Alphaviruses are enveloped single stranded RNA viruses that have a broad host range, and when used in gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1): 13-25; Lundstrom, Viruses. 2014 June; 6(6): 2392-2415; Lundstrom, Curr Gene Ther. 2001 May; 1(1):19-29; Rayner et al., Rev Med Virol. 2002 September-October; 12(5):279-96.

### Live Commensal HPV Vaccine Strategy

A live commensal HPV vaccine strategy can be used to optimally boost antiviral T cell immunity in the mucosa to prevent cancer development and treat early mucosal cancers or precancerous lesions with active virus, which include adenocarcinoma in situ, intraepithelial lesions of the vagina, penis and anus; squamous intraepithelial lesions (SILs); cervical intraepithelial neoplasia (CIN); and dysplasia and carcinoma in situ. Platforms to generate and expand live low-risk HPVs in culture in order to generate live and live attenuated HPV vaccine for use in patients are described in WO 2020/112720. Live HPV vaccines and live attenuated HPV vaccines, e.g., as described in WO 2020/112720 can be used.

### T Cell Adjuvant

The compositions can also include an adjuvant to increase T cell response. For example, nanoparticles that enhance T cell response can be included, e.g., as described in Stano et al., Vaccine (2012) 30:7541-6 and Swaminathan et al., Vaccine (2016) 34:110-9. See also Panagioti et al., Front. Immunol., 16 Feb. 2018; doi.org/10.3389/fimmu.2018.00276. Alternatively or in addition, an adjuvant comprising poly-ICLC (carboxymethylcellulose, polyinosinic-polycytidylic acid, and poly-L-lysine double-stranded RNA), Imiquimod, Resiquimod (R-848), CpG oligodeoxynuceotides and formulations (IC31, QB10), AS04 (aluminium salt formulated with 3-O-desacyl-4′-monophosphoryl lipid A (MPL)), AS01 (MPL and the saponin QS-21), MPLA, STING agonists, other TLR agonists, GM-CSF, Fms-like tyrosine kinase-3 ligand (Flt3L), and/or IFA (Incomplete Freund's adjuvant) can also be used. In some embodiments, topical imiquimod and/or topical 5-fluorouracil and/or topical calcipotriene (calcipotriol) in combination with 5-fluorouracil (e.g., as described in Cunningham et al., J Clin Invest. 2017; 127(1):106-116) could serve as adjuvants for the vaccine (this would be particularly applicable in subjects with pre-malignant lesions, who are commonly treated with these topical agents). See, e.g., Khong and Willem, Journal for ImmunoTherapy of Cancer 4:56 (2016); Coffman et al., Immunity. 2010 Oct. 29; 33(4): 492-503; Martins et al., EBioMedicine 3:67-78, 2016; and Del Giudice, Seminars in Immunology, 2018, doi.org/10.1016/j.smim.2018.05.001.

### Compositions

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, intratumoral, intramuscular or subcutaneous administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, intramuscular, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Subjects

The vaccine compositions described herein can be used to boost immunity against mucosal cancer in immunocompetent subjects, as well as immunosuppressed or immunocompromised patients who have reduced T cell immunity against β-HPVs and are prone to developing multiple mucosal warts and cancers (loaded with virus) with poor prognosis. In some embodiments, the subjects do not have cancer (e.g., do not have mucosal cancer). In some embodiments, the subjects are at high risk (i.e., have a risk that is above that of the general population) of developing mucosal cancer. For example, the subject may have a family history of cancer, a personal history of smoking, alcohol use, ionizing radiation, occupational exposures (e.g., acid mists), diet exposure (e.g., salted fish), precancerous lesions, relevant sexual history, or a family history or personal history of mucosal cancer. In some embodiments the subjects have, or have a history of, recurrent respiratory papillomatosis.

In some embodiments, the subject may be immunosuppressed, e.g., due to an organ transplant, an acquired immunodeficiency, e.g., HIV/AIDS, or primary human immunodeficiency.

Subjects who can be treated using the present methods include mammals, e.g., human and non-human veterinary subjects.

Methods of Inducing Anti-Cancer Immunity

The present compositions can be used to induce anti-cancer immunity, to reduce the risk of developing mucosal cancer, i.e., a cancer of a mucosal tissue. Mucosal cancers include cancers of the oral and sinonasal mucosa including mucosal squamous cell carcinoma (mSCC); head and neck cancer (HNC); cancers of the mucosa of the upper respiratory tract; and cancers of the genitourinary tract, including anal cancer, cervical cancer, and cancers of the vulva, vestibule, vagina, perineum and perianal. The methods include administering one or more doses of the vaccine compositions described herein to a subject, e.g., a subject in need thereof.

The compositions are administered in an effective amount. An “effective amount” is an amount sufficient to effect beneficial or desired results. For example, an effective amount is one that achieves a desired therapeutic effect, e.g., an amount necessary to treat a disease, or to reduce risk of development of disease or disease symptoms (also referred to as a therapeutically effective amount or a prophylactically effective amount, respectively). An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, the methods can include administering a first dose, followed by a second dose at a later time (e.g., a “booster” dose), e.g., at 1, 2, 4, 6, 8, 12, 18, 24, or 52 weeks later.

Dosage, toxicity and therapeutic efficacy of the therapeutic compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit high therapeutic indices are preferred. While compositions that exhibit toxic side effects may be used, care should be taken to minimize and reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compositions used in the methods described herein, the therapeutically effective dose can be estimated initially from cell culture assays. A dose may be formulated in animal models. Such information can be used to more accurately determine useful doses in humans.

The methods can also include administration of one or more other treatments known in the art for mucosal cancer, e.g., in subjects who have mucosal cancer, or treatment to reduce the risk of developing mucosal cancer. For example, a combination treatment with the compositions described herein plus treatments for benign or precancerous lesions (to reduce risk of developing mucosal cancer) (e.g., surgical treatments (e.g., excision (e.g., loop electrosurgical excision procedure (LEEP) or large loop electrosurgical excision of the transformation zone (LLETZ) for cervical lesions, or cold-steel surgery, laser excision and laser evaporation for vaginal lesions) or destruction (e.g., with carbon dioxide vaporization, cryotherapy, electrocauterization, or cold (thermo) coagulation)); photodynamic therapy; topical or systemic medications (e.g., podophyllin resin, podophyllotoxin, organic acids, such as salicylic acid, trichloroacetic acid and bichloroacetic acid, 5-fluorouracil, topical imiquimod, calcipotriene plus 5-fluorouracil, bleomycin, IFN-α, or cidofovir)), can be used in combination with the present methods). In some embodiments, these agents boost antigen presentation (innate signals) while the present compositions boost antigen recognition by T cells. In subjects who have mucosal cancer, surgical treatments (e.g., excision (e.g., loop electrosurgical excision procedure (LEEP) or large loop electrosurgical excision of the transformation zone (LLETZ) for cervical lesions, or cold-steel surgery, laser excision and laser evaporation for vaginal lesions) or destruction (e.g., with carbon dioxide vaporization, cryotherapy, electrocauterization, or cold (thermo) coagulation)); radiation therapy; photodynamic therapy; topical or systemic medications (e.g., podophyllin resin, podophyllotoxin, organic acids, such as salicylic acid, trichloroacetic acid and bichloroacetic acid, 5-fluorouracil, topical imiquimod, calcipotriene plus 5-fluorouracil, bleomycin, IFN-α, or cidofovir); or chemotherapy (e.g., with platinum-containing compounds such as cisplatin), can be used in combination with the present methods. See, e.g., IARC Working Group on the Evaluation of Carcinogenic Risks to Humans. Human Papillomaviruses. Lyon (FR): International Agency for Research on Cancer; 2007. (IARC Monographs on the Evaluation of Carcinogenic Risks to Humans, No. 90.) 1, Human Papillomavirus (HPV) Infection. Available from ncbi.nlm.nih.gov/books/NBK321770/

### EXAMPLES

The invention is further described in the following examples, which do not limit the scope of the invention described in the claims.

### Example 1

To broaden the application of commensal HPV vaccine for cancer prevention and therapy from the skin (e.g., as described in WO 2020/112720) to mucosal sites, we examined whether papillomavirus colonization of the oral cavity protects the immunocompetent hosts from mSCC.

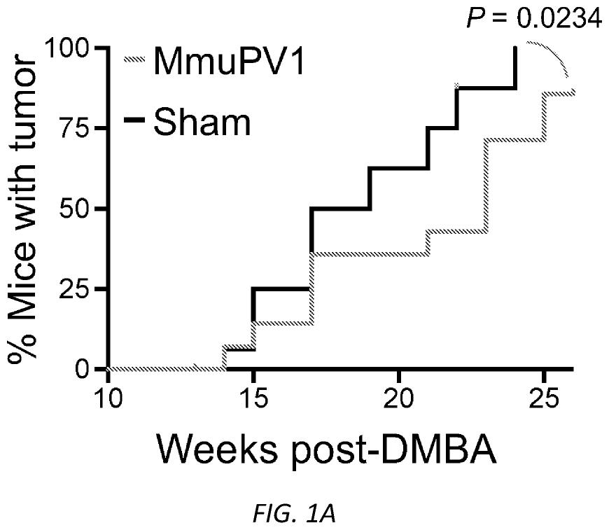

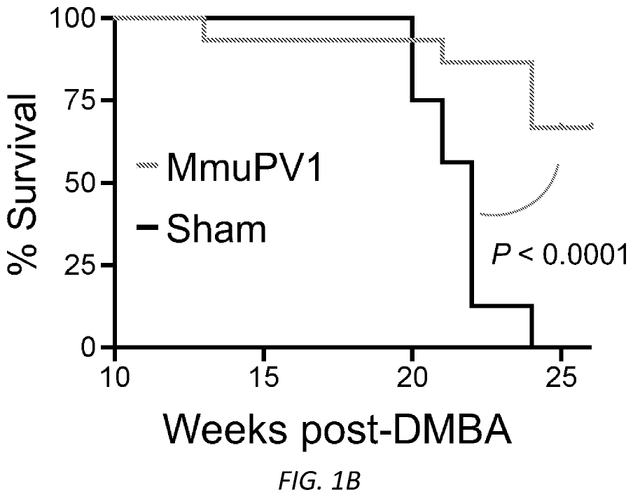

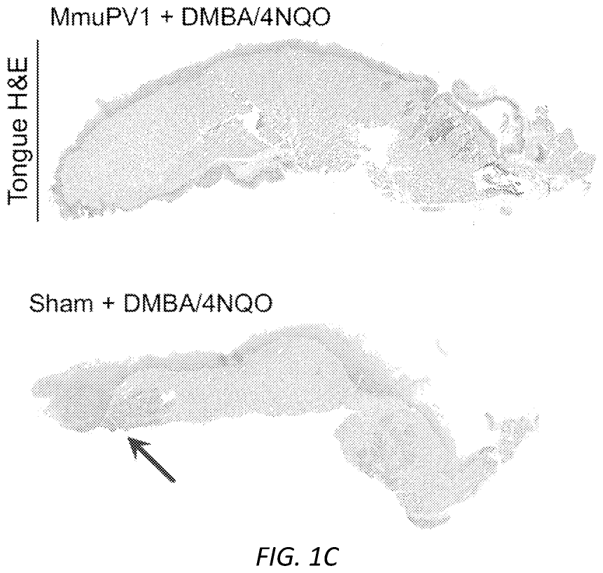

Method: We colonized the tongue of immunocompetent p53^{+/-} mice with MmuPV1, which does not result in any oral lesions. Briefly, mice were anesthetized with isoflurane followed by micro-aberrations over 0.2-0.3 cm² area on the base of the tongue using an 18-gauge sterile needle. Purified virus inoculum from MmuPV1-induced muzzle warts of B6.Cg-Foxn1^{nu/nu} mice was applied onto the scarified tongue and spread homogeneously. The same viral inoculum was used for all infected mice. The sham infection was performed with MmuPV1 VLPs (105 μg in 10 μL PBS per mouse) applied to the abraded tongue. Two weeks after MmuPV1 and sham oral infection, mice were subjected to DMBA/4NQO carcinogenesis protocol that we have optimized to induce carcinogenesis on the tongue of p53^{+/-} mice on the C57BL/6 background (FIGS. 1A-C)⁷. Animals' tongues were treated with a single dose of 50 μg 7,12-Dimethylbenz(a)anthracene (DMBA) in 50 μL of sesame oil applied to the tongue followed by 0.5% 4-nitroquinoline 1-oxide (4NQO) treatment in drinking water for 20 weeks. Mice were monitored for signs of oral cancer and harvested at 26 weeks post-DMBA or at any timepoint that they developed severe oral lesions hindering their ability to eat resulting in >10% body weight loss.

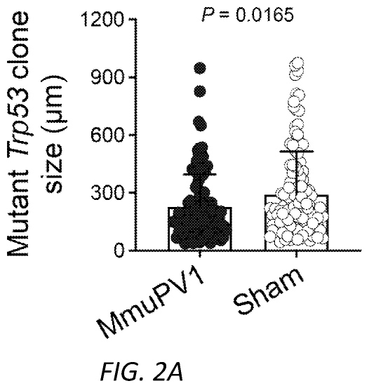

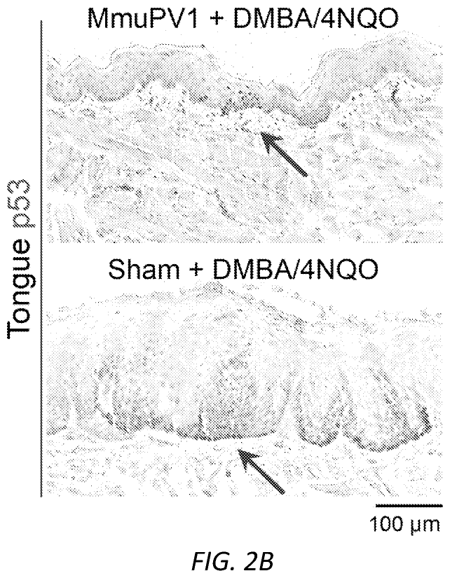

Result: The colonization of immunocompetent p53+/− mice with MmuPV1 protected the tongue from DMBA/4NQO-induced cancer compared with sham-infected controls. Mice that were colonized with MmuPV1 showed a significant delay in tongue tumor onset and survived marked longer compared with sham-infected animals (see FIGS. 1A-C). In addition, FIGS. 2A-B show the effect of papillomavirus tongue colonization on reducing the size of malignant clones (marked as Trp53 mutant clones) in mice tongue, which indicates the protective impact of papillomavirus colonization against early malignant transformation in mucosal epithelia. These findings substantiate the cancer-protective effect of commensal papillomavirus colonization in mucosal epithelia, which can be boosted by commensal HPV vaccines.

### REFERENCES

1. National Cancer Institute S, Epidemiology, and End Results Program Cancer Stat Facts: Common Cancer Sites.
2. Agalliu I, Gapstur S, Chen Z, Wang T, Anderson R L, Teras L, Kreimer A R, Hayes R B, Freedman N D, Burk R D. Associations of Oral alpha-, beta-, and gamma-Human Papillomavirus Types With Risk of Incident Head and Neck Cancer. JAMA Oncol 2016: 2; 599-606
3. Joseph A W, D'Souza G. Epidemiology of human papillomavirus-related head and neck cancer. Otolaryngol Clin North Am 2012: 45; 739-764
4. Bottalico D, Chen Z, Dunne A, Ostoloza J, McKinney S, Sun C, Schlecht N F, Fatahzadeh M, Herrero R, Schiffman M, Burk R D. The oral cavity contains abundant known and novel human papillomaviruses from the Betapapillomavirus and Gammapapillomavirus genera. The Journal of infectious diseases 2011: 204; 787-792
5. Ma Y, Madupu R, Karaoz U, Nossa C W, Yang L, Yooseph S, Yachimski P S, Brodie E L, Nelson K E, Pei Z. Human papillomavirus community in healthy persons, defined by metagenomics analysis of human microbiome project shotgun sequencing data sets. Journal of virology 2014: 88; 4786-4797
6. Strickley J D, Messerschmidt J L, Awad M E, Li T, Hasegawa T, Ha D T, Nabeta H W, Bevins P A, Ngo K H, Asgari M M, Nazarian R M, Neel V A, Jenson A B, Joh J, Demehri S. Immunity to commensal papillomaviruses protects against skin cancer. Nature 2019
7. Kim S. Animal models of cancer in the head and neck region. Clin Exp Otorhinolaryngol 2009: 2; 55-60

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating, or reducing the risk of developing, mucosal cancer in a subject, the method comprising administering to the subject an effective amount of a composition comprising:
a plurality of (i) antigenic peptides, each comprising a sequence of 9-30 amino acids derived from proteins from commensal human papilloma viruses, or (ii) live or live-attenuated commensal human papilloma viruses; and
a T cell adjuvant that increases T cell response to the antigenic peptides.

2. The method of claim 1, wherein the commensal human papilloma viruses are low risk α-HPV, β-HPV, γ-HPV, and/or μ-HPV strains.

3. The method of claim 1, wherein the commensal human papilloma viruses are β-HPV and/or γ-HPV strains listed in Table A.

4. The method of claim 1, wherein the plurality of antigenic peptides comprises peptides derived from one or more E1, E2, E4, E5, E6 or E7 proteins.

5. The method of claim 1, wherein the plurality of antigenic peptides comprises peptides derived from proteins from a plurality of commensal human papilloma viruses.

6. The method of claim 5, comprising at least 200 peptides each having a unique sequences.

7. The method of claim 6, comprising a plurality of peptides for each unique sequence.

8. The method of claim 1, wherein the T cell adjuvant comprises one or more of nanoparticles that enhance T cell response; poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminium salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant).

9. The method of claim 1, wherein the T cell adjuvant comprises topical imiquimod or topical 5-fluorouracil.

10. The method of claim 1, wherein the subject has an increased risk of developing mucosal cancer or is immunocompromised.

11. The method of claim 10, wherein the subject is immunocompromised as a result of an acquired immunodeficiency or an organ transplant.

12. A composition comprising:
a plurality of (i) antigenic peptides, each comprising a sequence of 9-30 amino acids derived from proteins from commensal human papilloma viruses, or (ii) live or live-attenuated commensal human papilloma viruses; and
a T cell adjuvant that increases T cell response to the antigenic peptides.

13. The composition of claim 12, wherein the subject has an increased risk of developing mucosal cancer or is immunocompromised.

14. The composition of claim 13, wherein the subject is immunocompromised as a result of an acquired immunodeficiency or an organ transplant.

15. The composition of claim 12, wherein the commensal human papilloma viruses are low risk α-HPV, β-HPV, γ-HPV, and/or μ-HPV strains.

16. The composition of claim 12, wherein the commensal human papilloma viruses are β-HPV and/or γ-HPV strains listed in Table A.

17. The composition of claim 12, wherein the plurality of antigenic peptides comprises peptides derived from one or more E1, E2, E4, E5, E6 or E7 proteins.

18. The composition of claim 12, wherein the plurality of antigenic peptides comprises peptides derived from proteins from a plurality of commensal human papilloma viruses.

19. The composition of claim 18, comprising at least 200 peptides each having a unique sequence.

20. The composition of claim 19, comprising a plurality of peptides for each unique sequence.

21. The composition of claim 12, wherein the T cell adjuvant comprises one or more of nanoparticles that enhance T cell response; poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminum salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant).

22. The composition of claim 12, wherein the T cell adjuvant comprises topical imiquimod or topical 5-fluorouracil.

23. The method of claim 1, wherein the mucosal cancer is a cancer of the oral and sinonasal mucosa, optionally mucosal squamous cell carcinoma (mSCC); head and neck cancer (HNC); a cancer of the mucosa of the upper respiratory tract; or a cancer of the genitourinary tract, optionally anal cancer, cervical cancer, or a cancer of the vulva, vestibule, vagina, perineum, or perianal tissue.
